(19) United States
(12) Patent Application Publication
(10) Pub. No.: US 2007/0287952 A1
(43) Pub. Date: Dec. 13, 2007

(54) MICRODIALYSIS PROBE

(76) Inventors: Jay P. Shah, Bethesda, MD (US); Terry Martyn Phillips, Washington, DC (US); Jerome V. Danoff, Bethesda, MD (US); Lynn Gerber, Bethesda, MD (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/741,451

(22) Filed: Apr. 27, 2007

Related U.S. Application Data
(60) Provisional application No. 60/795,176, filed on Apr. 27, 2006.

(51) Int. Cl. A61M 31/00

(52) U.S. Cl. 604/27; 604/28

(57) ABSTRACT

Provided are novel devices, systems, and methods for performing microanalysis of a localized biochemical milieu, and/or for highly localized drug delivery and treatment evaluation.

### CROSS REFERENCE TO RELATED APPLICATION

The present application claims priority to U.S. Provisional Patent Application No. 60/795,176, filed Apr. 27, 2006, the entire contents of which are incorporated herein by reference.

### FIELD OF THE INVENTION

The present application pertains to devices, systems, and methods for microanalytical evaluation of biological systems and delivery of therapeutic agents.

### BACKGROUND OF THE INVENTION

In vivo microdialysis is used to measure the chemical composition of interstitial fluid by means of a probe that includes a semi-permeable membrane. The inner surface of the probe membrane is perfused with physiological saline, and when the probe is implanted into tissue, molecules present in the interstitium diffuse across the membrane (down the concentration gradient) into the perfusion medium of the probe. Microdialysis enables in vivo sampling and measurement of tissue chemistry, and this technique has been applied to studies of human muscle, blood, adipose tissue, ocular tissues, brain, and liver. Microdialysis feasible in virtually every human organ, although limitations in currently-existing devices preclude certain uses. Exemplary microdialysis probes include the CMA/7 Microdialysis Probe (CMA Microdialysis AB, Solna, Sweden), the Eicom AZ Microdialysis Probe (Eicom, Kyoto, Japan), and the Applied Neuroscience MO-Hx microdialysis probe (Applied Neuroscience, London, UK), each of which consists of concentric outer and inner tubes, covered at the tip by a flexible dialysis fiber or membrane. A guide cannula is required to insert each of these microdialysis probe into the examination locus.

The ability to monitor the local biochemical milieu in soft tissue and organ systems could provide insights into the pathophysiology of musculoskeletal, neuromuscular, rheumatic, gastrointestinal, renal, cardiovascular, and endocrinologic diseases, as well as cancers, dermatological conditions, and pediatric disorders, for example, in premature newborns.

For example, pain is a complex process that involves the interaction of an array of biochemicals, transmitters, and receptors in both the central and peripheral nervous systems. The transformation of a tender nodule into a myofascial pain syndrome is poorly understood. However, local muscle pain is known to be associated with the activation of muscle nociceptors by a variety of endogenous substances including neuropeptides, arachidonic acid derivatives, and inflammatory mediators, among others (Mense S and Simons D G. Muscle Pain: Understanding Its Nature, Diagnosis and Treatment. Baltimore, Md.: Lippincott, Williams and Wilkins, 2001). Myofascial trigger points (MTrPs) are a very common cause of clinically observed local muscle pain and tenderness.

Elucidation of the underlying mechanisms of chronic neuropathic pain is providing clinicians with greater understanding of persistent neuropathic pain and treatment options (Moore K A, Baba H, and Woolf C J. Gabapentin—actions on adult superficial dorsal horn neurons. Neuropharmacology 43: 1077-1081, 2002; Yaksh T L. Future advances in pain pharmacology: what does the present say about the future? Proc West Pharmacol Soc 45: 211-218, 2002; Yaksh T L, Yamamoto T. and Myers R R. Pharmacology of nerve compression evoked hyperesthesia. In: Hyperalgesia and Allodynia, edited by Willis W. New York: Raven, 1995, p. 245-258). For example, in vivo and in vitro serological studies of peripheral blood and central nervous system (CNS) assays have shown cytokine tumor necrosis factor-α (TNF-α) to be critically involved in the pathogenesis of neuropathic pain states. In animal models, local TNF-α administration can evoke spontaneous electrophysiological activity in afferent C and A-delta nerve fibers that results in low-grade nociceptive input contributing to central sensitization. Anti-TNF-α agents reduce both the neuropathologic and behavioral manifestations of neuropathic pain states (Myers R R, Wagner R, and Sorkin L S. Hyperalgesia actions of cytokines on the peripheral nervous system. In: Cytokines and Pain: Progress in Inflammation Research, edited by Watkins L. Basel: Birkhauser Verlag, 1999, p. 133-158).

Nociceptive terminals in muscle display a multitude of different receptor molecules in their membranes, including matched receptors for well-documented endogenous substances such as bradykinin, 5-hydroxytryptamine (5-HT or serotonin), protons (H⁺), and prostaglandins that are released from damaged tissue. These biochemicals will bind with their matched receptors on the nociceptors bringing the membrane closer to threshold for an action potential. When summation is sufficient, action potentials will result, leading to local muscle pain and tenderness (Mense S and Simons D G, 2001). Furthermore, as bradykinin, 5-HT, and H⁺ sensitize the muscle nociceptors (i.e., lower than their normally high stimulation threshold), the sensitized muscle nociceptors are then more easily activated and may respond to normally innocuous and weak stimuli such as light pressure and muscle movement.

The continued presence of such biochemicals (and others) may be a necessary condition for persistent pain. However, little is known about the biochemical differences in the local tissue milieu between normal muscle and muscle with painful or nonpainful MTrPs, especially with respect to muscle contraction. Yet the pathophysiology, physical findings, and treatment methods of myofascial pain involve the local soft tissue (Mense S and Simons D G, 2001, Simons D G, et al., 1999).

Eliciting the local twitch response (“LTR”) via “dry needling” of MTrPs often produces a therapeutic benefit (Simons D G, et al., 1999). Local twitch response refers to the brief, local contraction that occurs when a trigger point is stimulated by snapping palpation or needle penetration. Dry needling refers to stimulation of a trigger point, usually with a narrow object such as a blunt needle or empty hypodermic needle. Local diagnostic methods such as thermography, EMG, biopsy, etc., have not elucidated the pathophysiology or shown changes after treatment. The elucidation of the pathophysiology of neurological conditions could aid the development of treatments targeted at those underlying mechanisms and to a better understanding of the relationship between relevant biological substances and the neuroplastic changes in the CNS that occur in, for example, chronic myofascial pain. However, devices and methods having the optimized characteristics that are ideal for performing such analysis have not yet been developed. Likewise, there does not yet exist an ideal means for in vivo measurement of bioavailable substances or drug substances directly from a wide variety of soft tissue and organ systems and suitable for all patient types.

### SUMMARY OF THE INVENTION

Provided are novel devices, systems, and methods for performing microanalysis of a localized biochemical milieu, and/or for highly localized drug delivery and treatment evaluation.

In one embodiment, there are provided microdialysis probes comprising a rigid housing having a lumen and a working end; an aperture in fluid communication with the lumen at said working end; a plurality of tubes extending longitudinally through said lumen, each of said plurality of tubes having a terminal opening, said terminal opening in fluid communication with the lumen near said working end; and, a semipermeable membrane disposed within said lumen and located between said aperture and said terminal openings.

In another embodiment, the inventive microdialysis probes comprise a hollow bore needle having a proximal end and a distal end, the proximal end including a substantially rounded hollow tip; a membrane disposed within said hollow bore needle at said proximal end; and, first and second tubing at least partially disposed within said hollow bore needle and in fluid communication with said membrane.

There are also provided microdialysis systems comprising a probe comprising a rigid housing having a lumen and a working end, an aperture in fluid communication with the lumen at said working end, a plurality of tubes extending longitudinally through said lumen, each of said plurality of tubes having a terminal opening, said terminal opening in fluid communication with the lumen near said working end, and, a semipermeable membrane disposed within said lumen and located between said aperture and said terminal openings; a pump in fluid communication with at least one of said plurality of tubes; and, a sample collector in fluid communication with at least one other of said plurality of tubes.

In other embodiments, the microdialysis systems can comprise a probe comprising a hollow bore needle having a proximal end and a distal end, the proximal end including a substantially rounded hollow tip, a membrane disposed within said hollow bore needle at said proximal end, and, first and second tubing at least partially disposed within said hollow bore needle and in fluid communication with said membrane; a pump in fluid communication with said first tubing; and, a sample collector in fluid communication with said second tubing.

Also provided are methods comprising contacting a tissue with a microdialysis probe, the probe comprising a rigid housing having a lumen and a working end; an aperture in fluid communication with the lumen at said working end; a plurality of tubes extending longitudinally through said lumen, each of said plurality of tubes having a terminal opening, said terminal opening in fluid communication with the lumen near said working end; and, a semipermeable membrane disposed within said lumen and located between said aperture and said terminal openings. In other embodiments, there are provided methods comprising contacting a tissue with a microdialysis probe, the probe comprising a hollow bore needle having a proximal end and a distal end, the proximal end including a substantially rounded hollow tip; a membrane disposed within said hollow bore needle at said proximal end; and, first and second tubing at least partially disposed within said hollow bore needle and in fluid communication with said membrane.

### BRIEF DESCRIPTION OF THE DRAWINGS

FIG. 1 provides a schematic of the basic construction an exemplary microdialysis probe.

FIG. 2 illustrates the positioning of an embodiment of the inventive microdialysis probe in the upper trapezius muscle of a subject, and the recorded EMG potential during a measurement of the local twitch response.

FIG. 3 depicts an exemplary microdialysis system that includes a Terasaki collection plate.

FIGS. 4A and 4B provide the pH level and concentration of bradykinin, respectively, as measured over time in “normal”, “active”, and “latent” subjects.

FIGS. 5A and 5B provide the concentrations of calcitonin gene-related peptide (CGRP) and substance P (SP), respectively, as measured over time in “normal”, “active”, and “latent” subjects.

FIGS. 6A and 6B provide the concentrations of tumor necrosis factor-α (TNF-α) and interleukin-1β (IL-1β), respectively, as measured over time in “normal”, “active”, and “latent” subjects.

FIGS. 7A and 7B provide the concentrations of serotonin and norepinephrine, respectively, as measured over time in “normal”, “active”, and “latent” subjects.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a tube” is a reference to one or more of such tubes and equivalents thereof known to those skilled in the art, and so forth. When values are expressed as approximations, by use of the antecedent “about,” it will be understood that the particular value forms another embodiment. As used herein, “about X” (where X is a numerical value) refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable.

Local diagnostic methods such as thermography, EMG, biopsy, etc., have not elucidated the pathophysiology of many conditions or shown local conditions after treatment thereof. Furthermore, existing microdialysis techniques involve the use of soft-tipped devices that cannot be introduced directly, e.g., without the use of a guide cannula, into soft tissue. Current devices cannot be precisely placed at or near a site of interest, cannot be accurately gauged for depth within tissue, and can invasively harm the tissue into which they are placed.

As described herein, superior microanalytical devices and systems has been developed to measure the in vivo biochemical milieu of sites of interest in near real-time at the subnanogram level of concentration. The devices and systems include a microdialysis probe capable of continuously collecting extremely small samples (˜0.5 μl) of physiological saline after exposure to the internal tissue milieu across a semipermeable membrane. Assaying the local milieu before, during, and after a pathophysiological condition can describe changes in bioactive substances that may contribute to the condition and to the effect of local treatment. The described microanalytical devices, systems, and techniques enable continuous sampling of extremely small quantities of substances directly from soft tissue, with minimal system perturbation and without harmful effects on subjects. The measured levels of analytes can be used, inter alia, to distinguish clinically distinct groups. The present invention provides minimally-invasive, highly versatile, rugged, microdialysis probes and systems. The instant probes and systems may also serve as in situ drug delivery vehicles of microdoses of medication to specific anatomical sites by slow diffusion. The invention also permits measurement of efficacy of drug delivery at the local tissue level, whether such drug has been administered orally, systemically, or topically. It can be utilized in a variety of patient populations and conditions. For example, the probe can be used to monitor the local biochemical milieu in soft tissue and organ systems to provide insights into the pathophysiology of musculoskeletal, neuromuscular, rheumatic, gastrointestinal, renal, cardiovascular and endocrinologic diseases, cancers, dermatological conditions, and pediatric disorders, including in connection with the delicate physiology of premature newborns.

In one embodiment, there are provided microdialysis probes comprising a rigid housing having a lumen and a working end; an aperture in fluid communication with the lumen at said working end; a plurality of tubes extending longitudinally through said lumen, each of said plurality of tubes having a terminal opening, said terminal opening in fluid communication with the lumen near said working end; and, a semipermeable membrane disposed within said lumen and located between said aperture and said terminal openings.

Also provided are microdialysis systems, comprising a microdialysis probe having a rigid housing having a lumen and a working end, an aperture in fluid communication with the lumen at said working end, a plurality of tubes extending longitudinally through said lumen, each of said plurality of tubes having a terminal opening, said terminal opening in fluid communication with the lumen near said working end, and, a semipermeable membrane disposed within said lumen and located between said aperture and said terminal openings; a pump in fluid communication with at least one of said plurality of tubes; and, a sample collector in fluid communication with at least one of said plurality of tubes.

As used herein, the term “rigid” is used to describe the quality of sufficient durability as to be capable of penetrating tissue without the assistance of a guide catheter or cannula. Accordingly, the rigid housing can be constructed from any material that can be inserted directly into tissue without the use of a cannula or other assisting device. Preferably, the rigid housing comprises a conventional needle, and is therefore constructed from stainless steel. The present microdialysis probes are preferably minimally invasive and, where used on living subjects, minimally painful to such subjects, and to this end a needle for use in supplying the rigid housing is ideally a high-gauge needle, such as a 32-gauge needle. Other rigid materials may also be used, including, for example, ceramic, plastic, or TEFLON. Any material that satisfactorily accommodates the described internal structures and that possesses sufficient rigidity may be used.

The working end of the rigid housing includes the portion of the instant microdialysis probes that, in use, can be inserted into the tissue. The aperture, which is disposed at the working end and is in fluid communication with the lumen of the rigid housing, is positioned within the tissue during use of the microdialysis probe, and such positioning places the lumen in fluid communication with the tissue.

Because the rigid housing can itself be inserted into tissue, it represents an advantageous design over traditional microdialysis probes, which feature “soft” tips. Traditional soft-tipped microdialysis probes cannot be inserted into tissue without the aid of a cannula or other guide device. Use of a cannula or other guide device renders the process of inserting a microdialysis probe more complex, more invasive, more time-consuming, more damaging to target tissue, less comfortable to patients, and less precise. The instant microdialysis probes, by contrast, are minimally invasive, induce less damage to the target tissue, are less irritating and painful to a patient, and can be positioned very precisely. Positioning is enhanced by the rigidity of the housing (a rigid probe is more manipulable and controllable than a flexible device), and in preferred embodiments, the housing is a high-gauge tube, which means that the device can be situated at a highly-specific locality with minimum tissue damage and discomfort to a patient. Precise positioning enables the delivery of drugs to a specific target within tissue, or the monitoring of biological processes at a highly-specific locality.

The precision of the inventive devices can be enhanced through the use of a depth regulator, monitor, or gauge, which is contrasted with traditional devices, that can only accommodate depth detection devices on the delivery cannula; because traditional microdialysis probes must be able to fit within a cannula, these probes cannot themselves accommodate a depth regulator, monitor, or gauge. In contrast, the instant probes can be fitted with depth detection devices, which enhances the precision of the probes themselves.

The working end of the rigid housing can comprise a substantially rounded tip. In preferred embodiments the substantially rounded tip can resemble the tip found on conventional acupuncture needles. A rounded tip can serve to minimize tissue injury and ensure patient comfort.

A plurality of “tubes” extend longitudinally through the lumen of the rigid housing. As used herein, a “tube” can refer to an independent structure that is distinct from the rigid housing and its lumen, or can refer to the space within the lumen that is not occupied by, for example, a separate independent tube. Thus, if an first independent tube is used and occupies half of the rigid housing lumen, the space within the lumen that is not occupied by the first tube can comprise a second “tube”.

Each tube has a terminal opening that is in fluid communication with a portion of the lumen near the working end of the rigid housing. As described above, the aperture is in fluid communication with the lumen, and positioning of the working end within tissue during use of the microdialysis probe places the lumen in fluid communication with the tissue; accordingly, such positioning also places the terminal opening of each tube in fluid communication with the tissue.

At the end of the rigid housing that is distal to the aperture, a portion of one or more of the plurality of tubes may extend beyond the lumen and into the open space beyond the rigid housing. At the end of the portion of a tube that extends into the open space beyond the rigid housing, the tube can be adapted for connection to a fluid flow line, a pump, or a sample collector. Accordingly, one or more of the plurality of tubes may be in fluid communication with one or more of a fluid flow line, a pump, or a sample collector. Alternatively, one or more of the plurality of tubes may extend within the lumen only to the end of the rigid housing that is distal to the aperture. In such instances, the end of the tube can be adapted for connection to one or more of a fluid flow line, a pump, or a sample collector. For example, a fluid flow line may be “plugged” directly through the end of the rigid housing that is distal to the aperture and into one of the plurality of tubes.

The instant microdialysis probes can further comprise a sheath. The sheath can partially or fully encircle the plurality of tubes at a location that is distal from the terminal openings of the tubes. For example, the sheath can encircle a portion of the tubes that extends beyond the lumen and into the open space beyond the rigid housing. In preferred embodiments, the sheath encircles a portion of the tubes that extends beyond the lumen and also encircles a portion of the rigid housing that is distal to the aperture.

One of the plurality of the tubes can function as an “inlet tube” that accommodates fluid flow from a source external to the rigid housing to the terminal opening of that tube, and therefore to the lumen and the aperture of the rigid housing, and to the tissue at or near which the rigid housing is positioned. Another of the plurality of tubes can function as an “outlet tube” that accommodates fluid flow from the lumen and/or aperture to the end of the rigid housing that is distal to the aperture; if the “outlet” tube extends beyond the end of the rigid housing that is distal to the aperture, then the tube can accommodate fluid flow from the lumen and/or aperture to a location external to the rigid housing.

An inlet tube can be used to receive saline from a fluid flow line or a pump and accommodate the flow of saline to the rigid housing lumen. The saline can then flow from the lumen to the aperture, and from the aperture to a site of interest (e.g., a tissue). An outlet tube can be used to receive fluid flow from the lumen, which can receive fluid that has traveled from the site of interest at or near which the probe has been positioned through the aperture. The fluid that has been received from the lumen into the outlet tube can flow to the end of the rigid housing lumen that is distal to the aperture, and from the outlet tube to a fluid flow line and/or a sample collector. The saline that can flow to the tissue via the inlet tube can in turn be used as a carrier fluid for biological material associated with the site of interest that can then be carried through the outlet tube an to a sample collector. Thus, the instant microdialysis probe is capable of cycling fluid from an outside source, to a site of interest such as a tissue, and to a location external to the rigid housing, such as a sample collector.

An inlet tube can also or alternatively be used to receive a fluid comprising a useful agent from a fluid flow line or pump and accommodate the flow of the fluid/useful agent to a site of interest. Fluid can then be delivered from the site of interest via an outlet tube to a location external to the rigid housing. The useful agent can be used to treat or otherwise have an effect on the site of interest, and the effects of the useful agent can be measured through analysis of the fluid received by a sample collector through an outlet tube. For example, a drug substance can be pumped through an external fluid flow line, into an inlet tube, through the rigid housing lumen and aperture, and to a tissue. Because the instant microdialysis probes are highly manipulable and capable of being precisely positioned at or near a site of interest, the drug substance in the preceding example can be delivered to a precise location rather than to a more generalized locality. Drug administration via the present microdialysis probes permits, for example, precise delivery directly to a cancerous tumor or microsatellites thereof, rather than via systemic administration or generalized, less precise local treatment.

In addition, because the inventive probes can be constructed using a very fine rigid housing, e.g., a high-gauge needle, a very small quantity of drug can be delivered per unit time. A continual infusion of drug, for example, over a period of time greater than 6 hours, greater than 10 hours, or greater than 12 hours can be achieved. Using conventional devices, a continual drip cannot be sustained for such extended periods of time.

Any effects on the site of interest resulting from the delivery of a useful agent via an inlet tube of the instant device can be measured through analysis of fluid that is withdrawn from the site of interest through the aperture and into an outlet tube, which may be in fluid communication with a sample collector. Following administration of a useful agent to a tissue, fluid from the local milieu of the tissue can be drawn into the sample collector, and this fluid can be analyzed in order to determine the effects (if any) of the useful agent on the local biochemical milieu of the tissue.

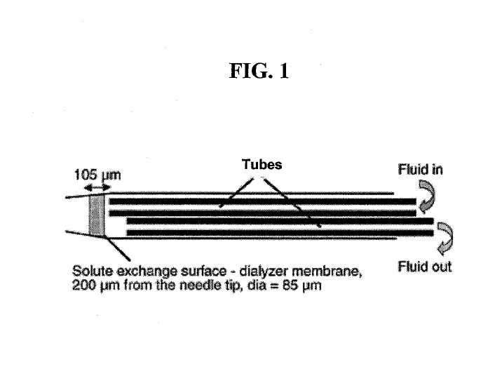

In preferred embodiments, the terminal openings of the respective tubes are longitudinally offset from one another (FIG. 1). This means that the terminal opening of one tube can be more proximate to the aperture than the terminal opening of a second tube. The offset distance, i.e., the distance between the respective terminal openings of two tubes, can be between about 75 μm and about 125 μm. A preferred offset distance is about 100 μm.

In the instant devices, a semipermeable membrane is disposed within the rigid housing lumen and is positioned between the aperture and the terminal openings of the plurality of tubes. Because the semipermeable membrane is disposed within the rigid housing, unlike in conventional devices, the membrane is protected from the forces that result from insertion of the microdialysis probe into tissue and manipulation of the probe within the tissue. The insertion and positioning capabilities of the instant probes are not limited by the presence of a “soft” external membrane portion, and the instant probes can be inserted into tissue, withdrawn, reinserted, and/or manipulated to the same degree as a conventional needle or acupuncture needle.

The semipermeable membrane may be circular, and can have a diameter that corresponds to the inner diameter of the rigid housing lumen. All fluid that enters the aperture must traverse the semipermeable membrane before it can contact the terminal openings of the tubes, and all fluid that exits the terminal openings of the plurality of tubes must traverse the semipermeable membrane before it can arrive at and/or pass through the aperture. The semipermeable membrane can be positioned about 150 μm to about 225 μm from the aperture, and about 70 μm to about 250 μm from the nearest tube's terminal opening. Preferably, the distance between the semipermeable membrane and the aperture is about 200 μm, and the distance between the semipermeable membrane and the nearest tube's terminal opening is about 100 μm.

The semipermeable membrane is preferably a dialysis membrane. Dialysis membranes are widely known among those skilled in the art. Nonlimiting examples of dialysis membranes include cellulose triacetate (CTA), polymethylmethacrylate (PMMA), polyacrylonitrile (PAN), polysulfone, nylon, and cellulose ester.

When the instant microdalysis probes are inserted into tissue, the tissue interstitium can enter the lumen via the aperture, and molecules present in the interstitium can (depending on their size) diffuse across the semipermeable membrane (down the concentration gradient) into a perfusion medium of the probe (e.g., saline, as delivered through the probe via an inlet tube). Microdialysis, the diffusion of molecules across the membrane, enables in vivo sampling and measurement of tissue chemistry. The semipermeable membrane can have a molecular cutoff of about 10 to about 150 kD, and in a preferred embodiment, the molecular cutoff is about 100 kD.

When the instant microdialysis probes are used for drug infusion, microdialysis via the semipermeable membrane can be used to determine the local effects of the drug on the site at which the probe is positioned. The probes can also be used to measure the concentration of drug at a site of interest (whether delivered via the probe or via alternative means, such as systemic administration), e.g., to determine whether the concentration of drug has reached a certain level at the site of interest.

In another embodiment, there are provided microdialysis probes comprising a hollow bore needle having a proximal end and a distal end, the proximal end including a substantially rounded hollow tip; a membrane disposed within said hollow bore needle at said proximal end; and, first and second tubing at least partially disposed within said hollow bore needle and in fluid communication with said membrane.

All fluid that enters the hollow bore needle at the tip must traverse the membrane before it can contact the tubing, and all fluid that exits the tubing, e.g., from an external source, must traverse the membrane before it can arrive at and/or pass through the tip. The membrane can be positioned about 150 μm to about 225 μm from the tip, and about 70 μm to about 250 μm from the nearest tubing's terminal opening. Preferably, the distance between the membrane and the tip is about 200 μm, and the distance between the membrane and the nearest tubing's terminal opening is about 100 μm.

Preferably, the hollow bore needle comprises a conventional needle, and is therefore constructed from stainless steel. The present microdialysis probes are preferably minimally invasive and, where used on living subjects, minimally painful to such subjects, and to this end a needle for use in supplying the hollow bore needle is ideally a high-gauge needle, such as a 32-gauge needle. Other rigid materials may also be used, including, for example, ceramic, plastic, or TEFLON. Any material that satisfactorily accommodates the described internal structures and that possesses sufficient rigidity may be used.

Each of the first and second tubing has an end that is in fluid communication with a portion of the hollow bore needle near the tip. The tip is in fluid communication with the hollow bore needle, and positioning of the probe within tissue can place the hollow bore in fluid communication with the tissue; accordingly, such positioning also places the end of each of the first and second tubing in fluid communication with the tissue.

In preferred embodiments, the respective ends of the first and second tubing that are disposed within the hollow bore needle are longitudinally offset from one another. This means that the end of the first tubing can be more proximate to the tip than the end of the second tubing. The offset distance, i.e., the distance between the respective ends of the first and second tubing, can be between 75 μm to about 125 μm. A preferred offset distance is about 100 μm.

The instant microdialysis probes can further comprise a sheath, and the sheath can surround the distal end of the hollow bore needle and at least a portion of the first and second tubing, e.g., a portion of the first and second tubing that is not disposed within the hollow bore needle.

Also provided are methods comprising contacting a tissue with any embodiment of the instant microdialysis probes. One of the tubes or tubing of the microdialysis probe may be in fluid communication with a pump, and a second tube or tubing may be in fluid communication with a sample collector. The instant methods may further comprise delivering a fluid via said pump through at least one tube or tubing in fluid communication with said pump. The fluid delivered through the at least one tube or tubing can comprise a beneficial agent, such as, for example, a drug.

In some instances, if a beneficial agent is delivered to a site of interest in accordance with the instant methods, it may be desirous to conduct an analysis of the site of interest, e.g., to monitor tissue drug levels or drug binding properties. The instant probes and methods permit the analysis of the effect of the beneficial agent concurrently with the delivery of the beneficial agent. Alternatively, after the microdialysis probe that was used to deliver the beneficial agent has been withdrawn, a second microdialysis probe can be placed at or near the site of interest, and analysis of the site of interest can be conducted by withdrawing fluid from the site of interest. Thus, the instant methods can further comprise withdrawing the initial probe, and contacting the tissue with a second microdialysis probe. The second microdialysis probe may be placed in fluid communication with a pump and with a sample collector via the probe's tubes or tubing. Fluid may be delivered to the site of interest from the pump via the tubes or tubing, and fluid from the site of interest may be withdrawn and delivered to a sample collector via another of the tubes or tubing.

Fluid that is withdrawn from a site of interest may be analyzed for the presence (including the quantity) of one or more analytes. Any analyte that is capable of diffusion through the membrane may be the subject of analysis, including, but not limited to hydrogen ions, cytokines, growth factors, neuropeptides, kinins, vasodilators, chemokines, neurotransmittors, hormones, and cell regulatory molecules.

### EXAMPLES

### Example 1

### Instrumentation

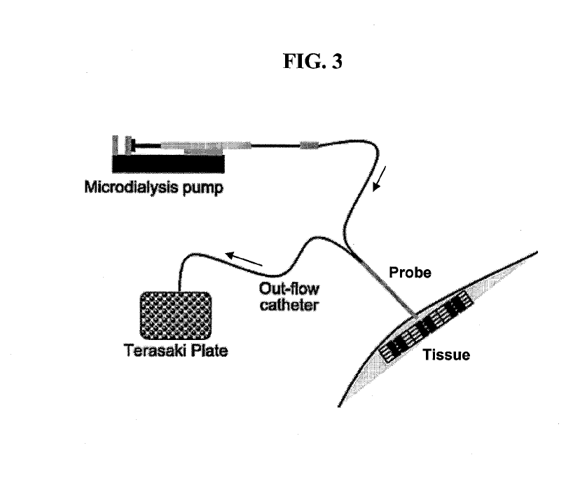

A prototype needle microdialysis system was developed comprising a hollow small-bore needle equipped with a microdialysis membrane, standard microdialysis connection tubing, a microdialysis perfusion pump, and a sample collection device. The system was designed to continuously collect samples, e.g., from the internal tissue milieu of human muscle, and could be used as a surrogate acupuncture needle during routine treatment of MTrPs (FIGS. 1 and 3).

Needle construction. The needle was constructed from a 2.5-in section of 30-gauge commercially available stainless steel hypodermic tubing (Small Parts, Miami Lakes, Fla.). One end was carefully ground internally and externally before being polished to a cone to minimize tissue injury in a similar manner to that of a standard Japanese-style acupuncture needle.

Membrane. An 85-μm-diameter disk was cut from a 105-μm-thick sheet of cellulose ester semi-permeable membrane (Spectrum Laboratories, Rancho Dominguez, Calif.) and placed on a specially designed jig. The jig enabled the precise positioning and cementing into place of the membrane exactly 200 μm from the ground tip of the needle.

Two extruded polyethylene tubes (Small Parts, Miami Lakes, Fla.) with the ends precisely offset by 100 μm from each other were glued together and positioned 200 μm from the membrane (FIG. 1). The dual-tube assembly was sealed into place and the proximal ends of the tubes were attached to 1 meter length standard fluoropolymer FEP microdialysis tubing via expandable tubing adaptors (0.12 mm-ID tubing; CMA Microdialysis, North Chelmsford, Mass.). The inlet tubing was attached to a model 102 microdialysis pump equipped with a 2.5-ml glass infusion syringe (CMA Microdialysis, North Chelmsford, Mass.), whereas the outlet tubing went to the sample collection device.

The syringe was loaded with 2.0 mL of sterile saline, pH 7.0, which was used to prime or flush the microdialysis needle. Collection of the dialysate was manually achieved by placing the end of the outlet tubing into a 20-μL well of a 72-well Terasaki microculture plate (Robbins Scientific, Sunnydale, Calif.) (FIG. 3). Consecutive samples were collected at predetermined time intervals by moving the catheter outlet from one well to another in an orderly manner. Each sample was collected under mineral oil to prevent loss by evaporation and to facilitate sample recovery and handling.

Characterization and calibration of the microdialysis system. The flow efficiency of the needle was tested by injecting a dilute dye through the needle and checking that the time taken for the dye to traverse the complete system was comparable with that calculated theoretically. Considering the length (˜1 m) of the FEP tubing at the inlet and outlet, it seemed reasonable to check flow and pressure. These were found to be within acceptable limits, and the needle was then subjected to further testing and calibration. The characteristics and efficiency of the microdialysis system were tested by placing the needle in a solution of saline containing commercially available molecular mass standards ranging from 1 to 150 kDa (Sigma-Aldrich, St. Louis, Mo.). The system was also tested for recovery and precision by repeatedly sampling the molecular mass standard solution and analyzing recoveries. Additionally, standard solutions of the 10 different analytes of interest were prepared in either saline or a soluble saline extract of postmortem (˜2 h postdeath with family consent) human muscle. The muscle extract solution was used to test the effects of muscle tissue fluid on analyte recovery. A mixture of equal parts of each analyte was prepared, and the microdialysis needle was used to sample the mixtures at flow rates of 1 μLl/min and 2 μL/min with samples collected at 0.5 and 0.25 min, respectively. Our Institutional Review Board did not approve in vivo calibration; therefore, the same mixture was deposited in defined areas of an isolated, perfused (rat trapezius) muscle. The needle was precisely inserted into these areas via a stereotactic positioner (Harvard Apparatus, Holliston, Mass.), and the deposits were sampled at identical rates as described for the fluid samples. All recoveries were performed in triplicate.

### Example 2

### Sample Analysis

Sample analysis. Due to the extremely small volumes collected from the microdialysis system (˜0.5 μL), analysis of each sample was performed in the Ultramicro Analytical Immunochemistry Resource (UAIR) by immunoaffinity capillary electrophoresis (ICE) and capillary electrochromatography (CEC). Measurement pH was made with a modified microcombination electrode in combination with an Orion model 370 pH meter (Thermo Electron, Woburn, Mass.) capable of making pH measurements in ˜0.2 μL of fluid. Samples were examined within 4 h of collection, and pH measurements were made immediately on arrival in the UAIR. Each sample was recovered from the Terasaki plate, measured for volume, and stored at −80° C. until analyte analysis by ICE and CEC. The major advantages of ICE and CEC are the extremely small sample volumes required for analysis (˜50 nl or 0.05 μL) and their high detection sensitivities (˜0.5 pg/mL). An added advantage of ICE is that the employment of an antibody-based initial extraction, followed by laser-induced fluorescence detection allows specific analytes to be isolated and measured in complex biological fluids; for example, the interstitial fluids surrounding the muscle trigger points.

ICE analysis of microdialysis samples. Measurement of the inflammatory cytokines (IL-1β, TNF-α), pain-associated neuropeptides [calcitonin gene-related peptide (CGRP), substance P (SP)], and bradykinin was performed as previously described (Phillips T M & Dickens B F. Analysis of recombinant cytokines in human body fluids by immunoaffinity capillary electrophoresis. Electrophoresis 19: 2991-2996, 1998; Phillips T M. Analysis of single-cell cultures by immunoaffinity capillary electrophoresis with laser-induced fluorescence detection. Luminescence 16: 145-152, 2001; Phillips T M. Determination of in situ tissue neuropeptides by capillary immunoelectrophoresis. Anal Chim Acta 372: 209-218, 1998). Briefly, antibody fragments (FAb) were prepared from antibodies specific to the analyte and immobilized onto 4 cm of the inner surfaces of a 100-μm ID fused silica capillary (Polymicro Technologies, Phoenix, Ariz.) via a disulfide linkage (Phillips T M & Dickens BF, 1998; Phillips T M, 2001; Phillips T M, 1998). The antibody-coated capillary was mounted into a Crystal 660 Capillary Electrophoresis system (Prince Technologies, Amsterdam, Netherlands) equipped with a Zetalif laser-induced fluorescence detector and a 12 mW 633 helium-neon laser (Picometrics, Ramonville, France). A 50-nL volume of collected sample was vacuum injected into the capillary and allowed to incubate with the immobilized antibodies for 5 min, during which each of the different antibodies interacted with and bound their specific analyte. Nonbound materials were flushed out of the capillary and collected for further analysis. The bound analytes were labeled in situ with AlexaFluor 633 laser dye (Molecular Probes, Eugene, Oreg.), flushed to remove nonreactive material, and the bound analytes were then electroeluted at pH 1.5. The individual analytes were separated by electrophoresis at 175 μA constant current, and the individual peaks were analyzed by the fluorescence detector using DAX peak area analytic program (Prince Technologies). All peak areas were then compared with those obtained by analyzing standard curves of each analyte.

CEC analysis of microdialysis samples. Measurement of serotonin and norepinephrine was performed on a Micro-Tech Scientific Ultra-Plus II MD CEC system (Micro-Tech Scientific, Vista, Calif.) equipped with a 100 mm×100 μm ID capillary column packed with 5 μm C₁₈ particles. A 20-nL volume of collected sample was injected by electrical migration into the system and processed at a constant voltage of 15 kV for 30 min according to a modification of the technique described by Oguri et al. (Oguri S, Yoneya Y. Mizunuma M, Fujiki Y. Otsuka K, and Terabe S. Selective detection of biogenic amines using capillary electrochromatography with an on-column derivatization technique. Anal Chem 74: 3463-3469, 2002). Detection of the individual analytes was achieved by filling the column with the amine coupling agent, o-phthalaldehyde-2-mercaptoethanol dissolved in borate buffer, pH 10, and allowing each separated analyte to react, via their free amino groups, with the o-phthalaldehyde to form fluorescent-labeled compounds. These products were detected online at 340 nm using the system's fluorescence detector. Areas under the peaks were analyzed and compared with known standards run under identical conditions.

### Example 3

### Results and Characterization of System Calibration

Recovery of the molecular mass standards demonstrated that the largest standard to be reliably recovered was 75,000 Da, with maximum recovery efficiency (<98%) in the molecular mass range of 0.5-45 kDa. The membrane was capable of being regenerated ˜50 times before recovery efficiency dropped below 90% and the system failed to recover the 75 kDa standard. Recoveries of the analytes of interest from both saline and human muscle extract at the two different flow rates are shown in Table 1, below, along with the recovery of the rat muscle-deposited analyte mixture. Although recoveries were acceptable for all analytes when sampled at 1 μl/min, recoveries at 2 μl/min demonstrated higher recoveries of all analytes, recoveries being >86% in both the fluid samples and the tissue deposits. Collected samples were examined for volume variation and the presence of air bubbles. The mean volume collected per patient was 0.5±0.003 μl and the presence of air bubbles was undetectable in any of the patient samples.

Table 1, below, depicts results from the recovery of analyte standards from saline, human muscle extract, and isolated rat trapezius muscle in-situ deposition.

TABLE 1

Percentage Recovery at
Different Flow Rates

| Analyte | Medium | 1 μl/min | 2 μl/min |
|---|---|---|---|
| Bradykinin | Saline | 85.6 ± 6.2 | 94.6 ± 3.6 |
| | Tissue extract | 86.6 ± 8.8 | 93.8 ± 5.4 |
| | In situ | 88.4 ± 7.2 | 95.0 ± 5.8 |
| CGRP | Saline | 95.5 ± 3.8 | 97.6 ± 2.1 |
| | Tissue extract | 94.9 ± 4.2 | 96.8 ± 3.5 |
| | In situ | 93.7 ± 5.5 | 96.2 ± 3.2 |
| SP | Saline | 95.6 ± 4.2 | 98.2 ± 1.2 |
| | Tissue extract | 94.9 ± 4.1 | 97.2 ± 2.2 |
| | In situ | 92.1 ± 5.6 | 96.3 ± 2.8 |
| TNF-α | Saline | 96.9 ± 2.8 | 98.1 ± 1.1 |
| | Tissue extract | 94.6 ± 3.9 | 96.3 ± 3.4 |
| | In situ | 92.5 ± 5.1 | 93.4 ± 5.3 |
| IL-1β | Saline | 97.8 ± 0.8 | 98.1 ± 0.6 |
| | Tissue extract | 96.9 ± 1.3 | 97.2 ± 1.4 |
| | In situ | 95.5 ± 1.8 | 96.7 ± 2.9 |
| Serotonin | Saline | 89.7 ± 9.3 | 92.0 ± 8.3 |
| | Tissue extract | 83 ± 16.3 | 89.3 ± 10.3 |
| | In situ | 84.7 ± 9.8 | 90.5 ± 9.0 |
| Norepinephrine | Saline | 92.3 ± 8.0 | 96.0 ± 3.8 |
| | Tissue extract | 84.0 ± 13.5 | 92.3 ± 7.0 |
| | In situ | 86.5 ± 10.3 | 89.8 ± 8.8 |

Values are means ±SD. Analyte standards are a standard mixture containing 40 nM serotonin and norepinephrine; 50 pM bradykinin, and 100 pg of substance P (SP) calcitonin gene-related peptide (CGRP), and the 4 cytokines at pH 7.4.

Both ICE and CEC are established analytic techniques known to yield reliable results. The antibodies used in the ICE technique were carefully selected based on no cross-reactivity with the other analytes of interest when tested by two-dimensional Western blotting and mass spectrometry. Absolute identification of the captured or isolated analytes was confirmed by matrix-assisted laser desorption time-of-flight mass spectrometry. The precision and accuracy of the analytical system was measured by running a standard sample five times on the same day and on 5 consecutive days. The intra-assay coefficients of variance (CV) for the panel of analytes was found to be <5.88±0.21, and the interassay CV was <6.04±0.31. Additionally, recoveries of analytes from standard solutions were shown to be >97.4% for all analytes. On the basis of these findings, we believed that these techniques would then be usable to distinguish the clinically distinct groups identified for this study.

### Example 4

### Use of Human Test Subjects

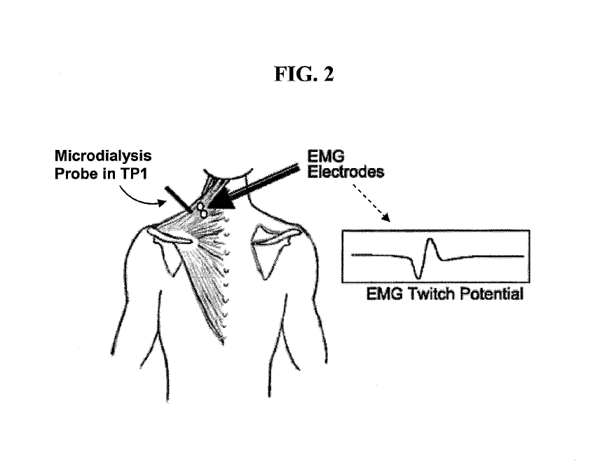

Eligible subjects were recruited from among clinical center staff. To be eligible, they completed a Brief Pain Inventory (BPI) and underwent a standard musculoskeletal examination by a single examiner, including palpation for MTrPs at trigger point 1 (TP1) (FIG. 2) (Simons D G, Travell J G, and Simons P T. Travell and Simons' Myofascial Pain and Dysfunction: The Trigger Point Manual. Vol. 1. Upper Half of Body (2nd ed.). Baltimore, Md.: Williams and Wilkins, 1999). Subjects were assigned to one of three groups based on history and physical examination: group 1) normal (no neck pain, no MTrP); group 2) latent (no neck pain, latent MTrP present); group 3) active (continuous idiopathic cervical pain of <3 month duration, MTrP present). The first three subjects to qualify for each group were used, yielding a total of nine subjects. Additional qualifiers were identified for later use in data gathering procedures.

Exclusion criteria for all groups included fibromyalgia, cervical radiculopathy, infection, cancer, history of local treatments or medications, history of smoking, and psychological issues such as fear of needles. This protocol was approved by an Institutional Review Board at the National Institutes of Health, and all subjects signed an Institutional Review Board-approved informed consent.

A pressure algometer (Pain Diagnostics and Treatment, Great Neck, N.Y.) was used to measure the local tenderness [pressure pain threshold (PPT)]. Pressure algometry was performed bilaterally on all subjects at TP1. Algometry procedures had been previously determined to be valid and reliable (Fischer A A. Algometry in diagnosis of musculoskeletal pain and evaluation of treatment outcome: an update. J Musculoskeletal Pain 6: 5-32, 1998).

The subject was comfortably positioned prone on a standard clinical plinth. Two pillows were used to support and stabilize the subject. Then the microdialysis needle was inserted into the upper trapezius muscle without penetrating the MTrP (if present). The needle remained in situ for 1 min before sample collection began. Dialysate was sampled according to the following schedule: every minute for the first 4 min, then every 10 s for 1 min (minute 4 to 5). At 5 min after needle insertion, the needle was advanced 1.5 cm deeper into the muscle until a LTR was obtained in groups 2 (latent) and 3 (active); a LTR was not observed in group 1 (normal). Recovery of dialysate continued every 10 s for the next 4 min and then every minute for 5 min. Total collection time was 14 min (4+1+4+5). Needle flow rate was maintained at 1 μL/min for the first 4 min and the final 5 min. For the middle 5 min (when samples were collected every 10 s), the flow rate was 2 μL/min.

To confirm the presence or absence of a LTR, simultaneous surface electromyography was performed using a Nicolet EMG Unit (Nicolet Instrument Technologies, Madison, Wis.) (FIG. 2).

Each sample (39 per patient) was analyzed by ICE, CCE, and micro-pH for the following analytes: hydrogen ion (indication of pH), bradykinin, CGRP, SP, TNF-α, IL-1β, serotonin, and norepinephrine.

Three microdialysis probes were fabricated for this study. They were gas-sterilized between uses.

Results

The active group had a lower PPT (P<0.08), which, although not a low enough probability to achieve classical significance (0.05), might suggest greater tenderness or sensitivity. Overall (i.e., for all 3 time levels combined, 2, 5, and 11 min after needle insertion), the amounts of bradykinin, CGRP, SP, TNF-α, IL-1β, serotonin, and norepinephrine were significantly higher in the active group than the other two groups (P<0.01 or better). Overall, pH was significantly lower in the active MTrP group than the other two groups (P<0.03). At peak time (5 min after the start of data collection when the needle was advanced and the latent MTrP and active MTrP groups demonstrated twitches), peak values of CGRP and SP were significantly different in all three groups (active>latent>normal, P<0.02). In the active MTrP group, the post (11 min after needle insertion and 6 min after needle advancement) values of SP and CGRP were significantly lower than the pre (2 min after needle insertion) and peak values (P<0.02). Specific statistical differences are summarized in Table 2, below. Only significant comparisons are shown.

| | 3 Times Combined: | |
|---|---|---|
| | Pre (2 min), Peak (5 min), | |
| Analyte | Post (11 min) | Peak (5 min) |
| pH (inv H⁺ | Active < latent, normal (P < 0.03)* | |
| concentration) | | |
| Bradykinin | Active > latent, normal (P < 0.01) | |
| CGRP | Active > latent, normal (P < 0.01) | Active > latent > |
| | | normal (P < 0.02) |
| SP | Active > latent, normal (P < 0.01) | Active > latent > |
| | | normal (P < 0.02) |
| TNF-α | Active > latent, normal (P < 0.001) | |
| IL-1β | Active > latent, normal (P < 0.001) | |
| Serotonin | Active > latent, normal (P < 0.01) | |
| Norepinephrine | Active > latent, normal (P < 0.01) | |

| Group, Analyte | Significance |
|---|---|
| Active, CGRP | Post < Pre, Peak (P < 0.02) |
| Active, SP | Post < Pre, Peak (P < 0.02) |

*Significance level.

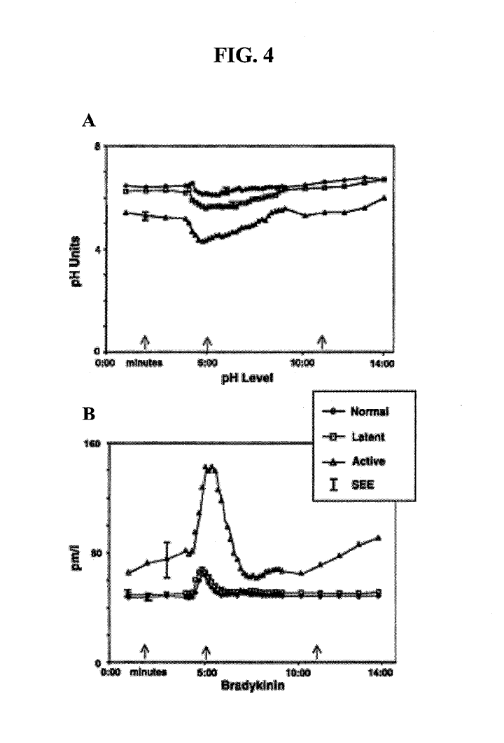

Graphs of the analytes averaged over all subjects and including all data points are depicted in FIGS. 4-7. Times used for the analysis (pre, peak, post) are indicated by vertical arrows, and the SEE (based on all data points) are indicated by vertical bars.

Recovery of the sample analytes is dependent on their concentrations in the local milieu and the physical parameters under which they are collected. Flow rate of the perfusate may have a major influence on the total collected solutes because diffusion is a rate-dependent property of the membrane-solution interface. If the flow rate is too rapid, insufficient contact time will be available for representative diffusion to take place. If the flow rate is too slow, gradients across the membrane may decrease in any local sample sufficiently to reduce diffusion. Previous work has demonstrated that flow rates in the range of 1 to 2 μl/min will allow a proportioned diffusion to take place, which can be used to calculate percentages of solutes in the local tissue milieu (Brown S A, Mayberry A J, Mathy J A, Phillips T M, Klitzman B, and Levin L S. The effect of muscle flap transposition on TNF-α levels during fracture healing. Plast Reconstr Surg 105: 991-998, 2000; Connelly C A. Microdialysis update: optimizing the advantages. J Physiol 514: 567-578, 1999; Hamin K, Rosdahl H, Ungerstedt U, and Henriksson J. Microdialysis in human skeletal muscle: effects of adding a colloid to the perfusate. J Appl Physiol 92: 385-393, 2002; Kehr J. A survey on quantitative microdialysis: theoretical models and practical implications. J Neurosci Methods 48: 251-261, 1993, Rosdahl H, Hamrin K, Ungerstedt U, and Henriksson J. A microdialysis method for the in situ investigation of the action of large peptide molecules in human skeletal muscle: detection of local metabolic effects of insulin. Int J Biol Macromol 28: 69-73, 2000).

Changes in flow rate are likely to account for some of the apparent increase in all analyte levels between 4 and 5 min. However, the increases up to the peak at 5 min did not achieve significance when compared with the initial levels as represented by data at the 2-min collection time. Another general observation is that for the active group, concentrations for all physical analytes appear to increase (although not statistically significantly) during the initial 4-min period when the needle was in the muscle. During this time the needle was close to, but not within, the MTrP. For the latent and control groups, the initial 4-min baselines were extremely stable. This may be indicative of a greater sensitivity to mechanical stimuli for the active group compared with the other groups, and this sensitivity may extend beyond the MTrP to relatively normal muscle tissue. Although these may not represent absolute levels of concentration, an important statistical finding is that for almost all comparisons, analyte levels of the active group are higher than the other groups (pH is lower).

Shortly after movement of the needle (5 min), analyte levels dropped. For the active group, not wishing to be bound by a particular theory, it is believed this is associated with chemical changes accompanying the muscle twitch. Why the other two groups also show decreases is not clear, but may be due to some similar local response associated with movement of the needle. All interpretations should be applied only to local conditions.

Previous studies using microanalytical needle techniques have used collection intervals ranging from 30 min to as much as several hours (e.g., Lott M E J and Sinoway L I. What has microdialysis shown us about the metabolic milieu within exercising skeletal muscle? Exerc Sport Sci Rev 32: 69-74, 2004). This is done to assure sufficient equilibration time for the concentrations in the perfusate to match the levels of interstitial analytes. Because equilibration is a time-based function, it was postulated that even if equilibration were not complete, proportionate relationships among different subject populations would still be identifiable. In addition, using long collection time intervals would be equivalent to smoothing a curve, which would tend to mask rapid changes in the measured analyte. It was desired to track changes more rapidly in analyte levels by using samples from relatively short collection intervals, which may offer insight into control mechanisms or temporal sequence of events. The ability to distinguish our different subject populations with short collection intervals appears to be supported by the results. Rapid changes in analyte levels may be identifiable.

The lower PPT found in subjects in the active group supports the well-known observation that people with active MTrPs are more sensitive to external mechanical stimuli. This may be due to general shifts in the nociceptor membrane potentials closer to action potential threshold, or it may be due to the presence of noxious metabolites that bombard and sensitize the membrane receptors. A combination of these two effects is also possible. However, in the current study, these PPT changes have only been observed locally, i.e., directly over the MTrP. Any changes in membrane sensitivities cannot be assumed to be generalized.

Differences in analyte concentration between the active group and the other two groups are not necessarily due to the MTrP. In fact, most differences were apparent from the time of needle insertion. These may be due to increased sensitivity of the active group to external stimulation (the needle occupying volume within the tissue). Some local changes are also to be expected with variations in blood flow, in particular bradykinin (Langberg H, Bjorn C, Boushel R, Hellsten Y, and Kjaer M. Exercise-induced increase in interstitial bradykinin and adenosine concentrations in skeletal muscle and peritendinous tissue in humans. J Physiol 542: 977-983, 2002). The active group might have different blood flow properties, and changes in blood flow (which were not recorded in this study) might alter membrane recovery properties or interstitial concentrations.

Several comparisons of analyte levels at the times representing pre, peak, and post levels appear to be different in the figures for the individual groups. However, statistically, these were not significant. Overall testing (3 times combined) achieved significance because the n (sample size) becomes effectively larger for combined data.

In the present study, subjects with active MTrPs and greater pain levels (i.e., pressure sensitivities) had lower pH levels in the vicinity of their MTrPs (FIG. 4A). A positive correlation has previously been shown between pain and local acidity (Issberner U, Reeh P W, and Steen K H. Pain due to tissue acidosis: a mechanism for inflammatory and ischemic myalgia? Neurosci Lett 208: 191-194, 1996). In a rat model, repeated injections of acidic saline into one gastrocnemius muscle produced bilateral, long-lasting mechanical hypersensitivity (i.e., hyperalgesia) of the paw (Sluka K A, Kalra A, and Moore S A. Unilteral intramuscular injections of acidic saline produce a bilateral, long-lasting hyperalgesia. Muscle Nerve 24: 37-46, 2001). The hyperalgesia was reversed by spinally administered μ- or ∂-opioid receptor agonists (Sluka K A, Rohlwing J J, Bussey R A, Eikenberry S A, and Wilken J M. Chronic muscle pain induced by repeated acid injection is reversed by spinally administered μ- and ∂-, but not κ-, opioid receptor agonists. J Pharmacol Exp Ther 302: 1146-1150, 2002) or N-methyl-D-aspartate (NMDA) or non-NMDA ionotropic glutamate receptor antagonists (Skyba D A, King E W, and Sluka K A. Effects of NMDA and non-NMDA ionotropic glutamate receptor antagonists on the development and maintenance of hyperalgesia induced by repeated intramuscular injection of acidic saline. Pain 98: 69-78, 2002). This model clearly demonstrates secondary mechanical hyperalgesia that is maintained by neuroplastic changes in the CNS. Furthermore, the persistent mechanical hyperalgesia was not caused by muscle tissue damage and was not maintained by continued nociceptive input from the site of injury (Sluka K A, et al., 2001). Therefore, an acidic milieu alone (without muscle damage) is sufficient to cause profound changes in the properties of nociceptors, axons, and dorsal horn neurons (i.e., the pain matrix). Mechanical hyperalgesia is a hallmark of a MTrP. An acidic pH is well known to stimulate the production of bradykinin during local ischemia and inflammation and may explain the cause of pain in patients with an active MTrP.

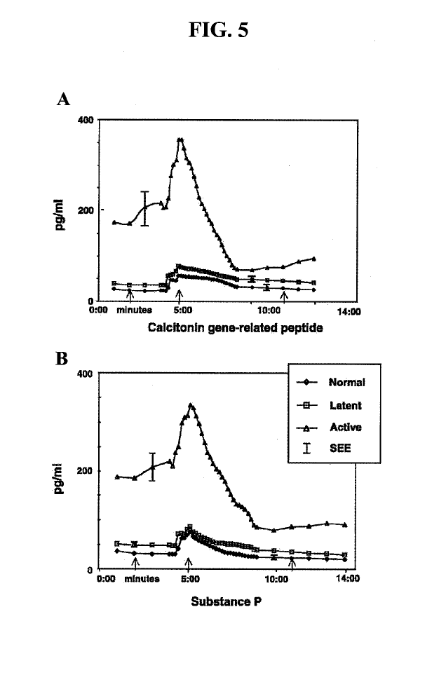

Significantly, elevated levels of SP and CGRP were observed in the vicinity of the active MTrPs (FIGS. 5A and 5B). SP and CGRP are produced in the dorsal root ganglion and over 90% of these biochemicals are transported antidromically down the neural process. There is a constant basal release of small amounts of these substances from the nociceptor into its local milieu (Yaksh T L, Yamamoto T. and Myers R R. Pharmacology of nerve compression evoked hyperesthesia. In: Hyperalgesia and Allodynia, edited by Willis W. New York: Raven, 1995, p. 245-258). However, their release is greatly increased in response to nociceptor activation (e.g., by H⁺ and bradykinin binding to their matched receptors). Small amounts of SP are also transported orthodromically from the dorsal root ganglion into the dorsal horn of the spinal cord. Prolonged nociceptor activation is known to greatly increase this process and directly lead to neuroplastic changes in the dorsal horn. This causes profound changes in neuronal activity and the perception of pain.

Both SP and CGRP dropped significantly after the muscle twitch. This concurs with the commonly observed (at least temporary) decrease in pain after the “release” of a MTrP.

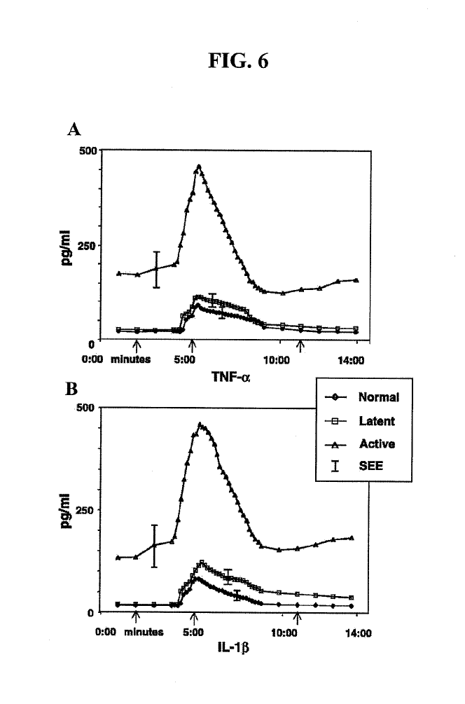

Significantly elevated levels of TNF-α and IL-1β were observed in subjects with active MTrPs (FIGS. 6A and 6B). In a rat model, TNF-α produces a time- and dose-dependent muscle hyperalgesia within several hours after injection into the gastrocnemius or biceps brachii. This hyperalgesia was completely reversed by systemic treatment with the nonopioid analgesic metamizol (Schafers M, Sorkin L S, and Sommer C. Intramuscular injection of tumor necrosis factor-alpha induces muscle hyperalgesia in rats. Pain 104: 579-588, 2003). Furthermore, TNF-α did not cause histopathological tissue damage or motor dysfunction. One day after injection of TNF-α, elevated levels of CGRP, nerve growth factor (NGF), and PGE₂ were found in the muscle. The present studies permit the conclusion that TNF-α and other proinflammatory cytokines such as IL-1β may play a role in the development of muscle hyperalgesia, and the targeting of pro-inflammatory cytokines might be beneficial for the treatment of muscle pain syndromes (Schafers M et al., 2003).

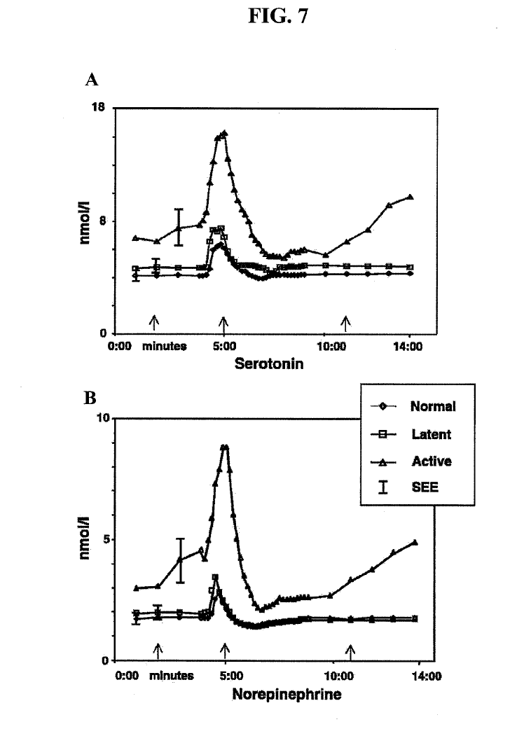

SP causes mast cell degranulation with the release of serotonin (in addition to histamine) and upregulation of proinflammatory cytokines. Increases in TNF-α stimulate the production of norepinephrine. Significantly elevated levels of serotonin and norepinephrine were observed in subjects with active MTrPs (FIGS. 7A and 7B). The increased levels of norepinephrine may be associated with increased sympathetic activity in the motor end plate region, which could then account for the lower threshold of a LTR.

The present minimally-invasive probes function as localized sampling tools and safely gather large amounts of data related to complex physiological events such as myofascial pain. The present devices are also sensitive enough to identify changes across conditions and clinical states.

The inventive microdialysis probes and systems, capable of using samples of <1 μl, provides continuous, real time, in vivo recovery of molecules 75 kDa and smaller directly from the soft tissue environment without harmful effects on subjects. Subsequent analysis of the collected samples can distinguish analyte levels before, during, and after a biochemical event of interest. For example, differences have been demonstrated in the level of measured analytes between people who have pain and those who do not and between those who have active MTrPs versus those who have latent or no MTrPs. The local milieu does appear to change with the occurrence of the LTR, and these changes were tracked with short (minutes) collection intervals.

What is claimed:

1. A microdialysis probe comprising:
a rigid housing having a lumen and a working end;
an aperture in fluid communication with the lumen at said working end;
a plurality of tubes extending longitudinally through said lumen, each of said plurality of tubes having a terminal opening, said terminal openings being in fluid communication with the lumen near said working end; and,
a semipermeable membrane disposed within said lumen and located between said aperture and said terminal openings.

2. The microdialysis probe of claim 1 wherein the terminal openings of the respective tubes are longitudinally offset from one another.

3. The microdialysis probe of claim 2 wherein the terminal openings are longitudinally offset by about 75 μm to about 125 μm.

4. The microdialysis probe of claim 1 wherein said semipermeable membrane is about 70 μm to about 250 μm from the nearest terminal opening of said plurality of tubes.

5. The microdialysis probe of claim 1 wherein said semipermeable membrane is about 150 μm to about 225 μm from the aperture.

6. The microdialysis probe of claim 1 wherein said semipermeable membrane comprises a 100 kDa microdialysis membrane.

7. The microdialysis probe of claim 1 wherein said rigid housing comprises a needle.

8. The microdialysis probe of claim 1 wherein said working end of said rigid housing comprises a substantially rounded tip.

9. The microdialysis probe of claim 1 further comprising a sheath that at least partially encircles said plurality of tubes at a location distal from said terminal openings.

10. The microdialysis probe of claim 1 wherein one or more of said plurality of tubes are adapted for connection to a fluid flow line.

11. A microdialysis probe comprising:
a hollow bore needle having a proximal end and a distal end, the proximal end including a substantially rounded hollow tip;
a membrane disposed within said hollow bore needle at said proximal end; and
first and second tubing at least partially disposed within said hollow bore needle and in fluid communication with said membrane.

12. The microdialysis probe of claim 11 wherein said membrane is disposed at a distance of up to 200 μm from said tip.

13. The microdialysis probe of claim 11 further comprising a sheath.

14. The microdialysis probe of claim 13 wherein said sheath surrounds the distal end and at least a portion of the said first and second tubing.

15. A microdialysis system comprising:
a probe, comprising
a rigid housing having a lumen and a working end;
an aperture in fluid communication with the lumen at said working end;
a plurality of tubes extending longitudinally through said lumen, each of said plurality of tubes having a terminal opening, said terminal openings being in fluid communication with the lumen near said working end; and,
a semipermeable membrane disposed within said lumen and located between said aperture and said terminal openings;
a pump in fluid communication with at least one of said plurality of tubes; and,
a sample collector in fluid communication with at least one other of said plurality of tubes.

16. The microdialysis system of claim 15 wherein the terminal openings of the respective tubes are longitudinally offset from one another.

17. The microdialysis system of claim 16 wherein the terminal openings are longitudinally offset by about 75 μm to about 125 μm.

18. The microdialysis system of claim 15 wherein said semipermeable membrane is about 70 μm to about 250 μm from the nearest terminal opening of said plurality of tubes.

19. The microdialysis system of claim 15 wherein said semipermeable membrane is about 150 μm to about 225 μm from the aperture.

20. The microdialysis system of claim 15 wherein said semipermeable membrane comprises a 100 kDa filter.

21. The microdialysis system of claim 15 wherein said rigid housing comprises a needle.

22. The microdialysis system of claim 15 wherein said working end comprises a substantially rounded tip.

23. The microdialysis system of claim 15 further comprising a sheath that at least partially encircles said plurality of tubes at a location distal from said terminal openings.

24. The microdialysis system of claim 15 wherein one or more of said plurality of tubes are adapted for connection to a fluid flow line.

25. A method comprising:
contacting a tissue with a microdialysis probe, said probe comprising
a rigid housing having a lumen and a working end;
an aperture in fluid communication with the lumen at said working end;
a plurality of tubes extending longitudinally through said lumen, each of said plurality of tubes having a terminal opening, said terminal openings being in fluid communication with the lumen near said working end; and,
a semipermeable membrane disposed within said lumen and located between said aperture and said terminal openings;
a semipermeable membrane disposed transversely within said internal space, located distally from said aperture and proximally from each of the terminal openings of said plurality of lumens.

26. The method according to claim 25 wherein at least one of said plurality of tubes is in fluid communication with a pump, and at least one other of said plurality of tubes is in fluid communication with a sample collector.

27. The method according to claim 26 further comprising withdrawing a fluid from said tissue through said at least one tube in fluid communication with a sample collector.

28. The method according to claim 27 further comprising analyzing said withdrawn fluid.

29. The method according to claim 28 wherein said analyzing said withdrawn fluid comprises measuring the presence of one or more analytes.

30. The method according to claim 29 wherein said one or more analytes are selected from hydrogen ions, cytokines, growth factors, neuropeptides, kinins, vasodilators, chemokines, neurotransmittors, hormones, and cell regulatory molecules.

31. The method according to claim 26 further comprising delivering a fluid via said pump through said at least one tube in fluid communication with said pump.

32. The method according to claim 31 wherein said fluid comprises a beneficial agent.

33. The method according to claim 32 further comprising withdrawing said probe, and contacting said tissue with a second microdialysis probe, wherein at least one of said plurality of tubes of said second microdialysis probe is in fluid communication with a pump, and at least one of said plurality of tubes of said second microdialysis probe is in fluid communication with a sample collector.

34. The method according to claim 33 further comprising withdrawing a fluid from said tissue through said at least one tube in fluid communication with a sample collector.

35. The method according to claim 34 further comprising analyzing said withdrawn fluid.

36. The method according to claim 35 wherein said analyzing said withdrawn fluid comprises measuring the presence of one or more analytes.

37. The method according to claim 36 wherein said one or more analytes are selected from hydrogen ions, cytokines, growth factors, neuropeptides, kinins, vasodilators, chemokines, neurotransmittors, hormones, and cell regulatory molecules.
